(19) United States
(12) Patent Application Publication
(10) Pub. No.: US 2007/0019159 A1
(43) Pub. Date: Jan. 25, 2007

(54) Deforming method for deformable mirror, aberration compensation method for optical apparatus, aberration compensation method of ocular fundus observation apparatus, aberration compensation apparatus, optical apparatus and ocular funds observation apparatus

(76) Inventors: Michiko Nakanishi, Tokyo (JP); Hiroyuki Kawashima, Tokyo (JP); Noriko Saito, Tokyo (JP)

Correspondence Address: KUBOVCIK & KUBOVCIK, SUITE 710, 900 17TH STREET NW, WASHINGTON, DC, 20006, US

(21) Appl. No.: 11/430,204

(22) Filed: May 9, 2006

(30) Foreign Application Priority Data
Jul. 20, 2005 (JP) 2005-210680
Jul. 20, 2005 (JP) 2005-210676
Jul. 20, 2005 (JP) 2005-210677

(51) Int. Cl. A61B 3/14

(52) U.S. Cl. 351/206

(57) ABSTRACT

A deforming method for a deformable mirror having plural electrodes and a reflective membrane which are distorted by static voltages. The Zernike voltage template used to deform a deformable mirror is adapted for aberration correction by calibrating each component template according to the specificity of the mirror. The voltages applied to the electrodes to form a surface profile of the reflective membrane are measured based on the reflection light from the membrane. A certain number of voltage-by-electrode patterns each of which corresponds to a different reference profile of the reflective membrane and specifies each set of a voltage and an electrode to which the voltage is to be applied are stored preliminarily. The reflective membrane is deformed to a desired profile by superposing preliminarily stored voltage-by-electrode patterns. The deformation of the mirror is corrected by calibrating each template according to the operational environment.

### BACKGROUND OF THE INVENTION

The present invention relates to a method of deforming a deformable mirror to correct the aberration of reflection light from a subject, an aberration compensation method in optical apparatus and the same in ocular fundus observation apparatus. In addition, the present invention relates to aberration correction apparatus, optical apparatus and ocular fundus observation apparatus which use deformable mirrors.

In conventional optical apparatus, the deformable mirror is used to correct the optical distortion of reflection light from a subject. Refer to Japanese Patent Laid-open Nos. 2004-247947 and 9-152505.

Clear ocular fundus images cannot be obtained in ocular fundus observation apparatus and the like, since reflection light from eyeground contains aberration due to the imperfect optical system of the eye. This aberration can be corrected by a deformable mirror. Refer to Japanese Patent Laid-open No. 11-137522. In the disclosed ocular fundus observation apparatus, a light flux emitted from an image pickup light source is incident on the eye of the person under inspection and the reflection light flux from the eyeground is guided into the recording means as image pickup light to record the image of the person's eyeground.

Typically, as shown in FIGS. 9 and 10, a deformable mirror 300 is disposed in the atmosphere of the room and comprises a membrane mirror 301 held tightly by a frame 302 and plural electrodes 305 formed at a certain distance from the membrane mirror 301. The membrane mirror 301 is a flexible membrane with a top surface for reflection and the plural electrodes (five electrodes 305-1-305-5 in this example) are formed on top of a flat substrate 304. In this example, each of the electrodes 305-1-305-5 is connected to a power supply 306 which can apply certain voltages V1-V6 respectively to the electrodes 305-1-305-5 of the conventional example. Certain voltages are respectively applied to the electrodes 305 so that part of the membrane mirror which faces each electrode is attracted by an electrostatic force which depends on the voltage V applied to the electrode and the distance between the membrane mirror and the electrode. Consequently, the membrane mirror 301 is distorted or deformed as desired. Note that numeral 307 refers to spacers to secure an amount of space between the membrane mirror 301 and the substrate 304. This deformable mirror 300, as shown in FIG. 10, is deformed to a desired profile by applying certain voltages V1-V5 to electrodes 305-1-305-5, respectively.

Further in “Compensation of model eye's aberration by using deformable mirror” (Proceedings of SPIE, MEMS/MOEMS Components and Their Applications II, Volumes 5717, p. 219-229, 2005), a deforming method for a deformable mirror which comprises plural electrodes and a reflective membrane faced to the plural electrodes and deformed to a certain profile by static voltages applied to the plural electrodes to correct the wavefront distortion of a light flux incident on the reflective membrane is disclosed with optical apparatus and ocular fundus observation apparatus using this method. To attain a desired deformation of the deformable mirror, this known method comprises the steps of: measuring the respective voltages applied to the electrodes to form a desired surface profile of the reflective membrane based on the detected reflection light from the reflective membrane; preliminarily storing a certain number of voltage-by-electrode patterns each of which corresponds to a different reference profile of the reflective membrane and specifies each set of a voltage and an electrode to which the voltage is to be applied; and deforming the reflective membrane to a desired profile by mutually superposing the preliminarily stored voltage-by-electrode patterns.

The above-mentioned deformable mirror has some specificity in terms of surface profile and deforming characteristics due to the manufacture/assembly and the residual stress in the mirror made of a SOI wafer. In addition, the deforming performance of the mirror varies due to the adjustment error which is inevitable when the total system is set up including the deformable mirror. Further, the surface profile and deforming characteristics of the mirror change as the operational environment changes in humidity, temperature and pressure. Therefore, in the case of the above-mentioned method comprising the steps of: measuring the respective voltages applied to the electrodes to form a desired surface profile of the reflective membrane based on the detected reflection light from the reflective membrane; preliminarily storing a certain number of voltage-by-electrode patterns each of which corresponds to a different reference profile of the reflective membrane and specifies each set of a voltage and an electrode to which the voltage is to be applied; and deforming the reflective membrane to a desired profile by mutually superposing preliminarily stored voltage-by-electrode patterns, the initial stored values can not be used to deform the mirror to a desired profile after the mirror changes its surface profile and the deforming characteristics. In the output signal, components are mixed with each other.

For example, if a Zernike voltage template is used to deform a deformable mirror in an ocular fundus observation apparatus or the like, a single-order Zernike voltage template can not be calculated by using other-order Zernike voltage templates. Since the mirror can not be deformed to a desired profile if the Zernike voltage template used to deform the mirror contains other components, accurate aberration correction is not possible.

Accordingly, it is an object of the present invention to provide a deformable mirror deforming method which allows an ocular fundus observation apparatus or the like to adapt the Zernike voltage template used to deform the deformable mirror for aberration correction by calibrating other-order Zernike voltage templates according to the varying specificity of the deformable mirror which is due to the operational environment, manufacturing error, etc. Note that “voltage template” in this specification means a table for voltage correction. In addition, “calibration” in this specification means adjustment or modification.

### SUMMARY OF THE INVENTION

According to an aspect of the present invention, there is provided a deforming method for a deformable mirror which comprises a plurality of electrodes and a reflective membrane faced to the electrodes and deformed to a certain profile by static voltages applied to the electrodes to correct wavefront distortion of a light flux incident on the reflective membrane. This deforming method comprises the steps of: preliminarily storing a certain number of voltage-by-electrode patterns each of which corresponds to a different reference profile of the reflective membrane and specifies each set of a voltage and an electrode to which the voltage is to be applied; deforming the reflective membrane to a desired profile by mutually superposing said preliminarily stored voltage-by-electrode patterns; and calibrating the deformation of the reflective membrane by correcting a certain number of the superposed voltage-by-electrode patterns.

According to another aspect of the present invention, there is provided a deforming method of a deformable mirror which comprises plural electrodes and a reflective membrane faced to the plural electrodes and deformed to a certain profile by static voltages applied to the plural electrodes to correct the wavefront distortion of a light flux incident on the reflective membrane. This deformable mirror deforming method comprises the steps of: preliminarily storing a certain number of voltage-by-electrode patterns each of which corresponds to a different reference profile of the reflective membrane and specifies each set of a voltage and an electrode to which the voltage is to be applied; deforming the reflective membrane to a desired profile by mutually superposing said voltage-by-electrode patterns stored preliminarily; comparing a preliminarily stored detection signal with a detection signal detected actually later; and calibrating the deformation of the reflective membrane by correcting the stored detection signal based on the actual operational condition of the deformable mirror so that a desired detection signal is obtained.

According to the present invention, the above-mentioned actual operational condition of the deformable mirror may include at least one of the deformable mirror's manufacturing error, comprehensive setup error, ambient humidity, temperature and pressure.

According to the present invention, the above-mentioned reference profiles of the reflective membrane may be provided respectively in association with certain-order elements of Zernike polynomials.

According to another aspect of the present invention, there is provided an aberration compensation method of an optical apparatus which comprises a plurality of electrodes and a reflective membrane faced to the electrodes and deformed to a certain profile by static voltages applied to the electrodes, wherein either of the above-mentioned deformable mirror deforming methods is performed as the aberration compensation method to correct the wavefront distortion of the light flux incident on the reflective membrane of the optical apparatus.

According to another aspect of the present invention, there is provided an aberration compensation method of an ocular fundus observation apparatus which includes a deformable mirror comprising a plurality of electrodes and a reflective membrane faced to the electrodes and deformed to a certain profile by static voltages applied to the plural electrodes, wherein either of the above-mentioned deformable mirror deforming methods is performed as the aberration compensation method to correct the wavefront distortion of the light flux incident on the reflective membrane of the optical apparatus.

According to another aspect of the present invention, there is provided an aberration correction apparatus, comprising: a deformable mirror which comprises a plurality of electrodes and a reflective membrane faced to the electrodes and corrects the wavefront distortion of a reflected light flux incident on the reflective membrane which is deformed to a certain shape by applying static voltages to the electrodes; a storage section to store a certain number of voltage-by-electrode patterns each of which corresponds with a different reference profile of the reflective membrane and specifies each set of a voltage and an electrode to which the voltage is to be applied; an aberration detecting section to detect the aberration of light which originates from a subject and is reflected from the deformable mirror; and calculation means for correcting the voltage-by-electrode patterns each of which is stored in the storage section, corresponds with a different reference profile of the reflective membrane and specifies each set of a voltage and an electrode to which the voltage is to be applied.

According to another aspect of the present invention, there is provided an aberration correction apparatus, comprising: a deformable mirror which comprises a plurality of electrodes and a reflective membrane faced to the electrodes and corrects the wavefront distortion of a reflected light flux incident on the reflective membrane which is deformed to a certain shape by applying static voltages to the electrodes; a storage section to store a certain number of voltage-by-electrode patterns each of which corresponds with a different reference profile of the reflective membrane and specifies each set of a voltage and an electrode to which the voltage is to be applied; an aberration detecting section to detect the aberration of light which originates from a subject and is reflected from the deformable mirror; and correction value calculation means for comparing a detection signal preliminarily stored in the storage section with an actual detection signal detected later to calculate correction values, wherein, the correction values are used to correct the stored detection signal based on the actual operational condition of the deformable mirror so that a desired detection signal is obtained.

According to the present invention, the above-mentioned actual operational condition of the deformable mirror may include at least one of the deformable mirror's manufacturing error, comprehensive setup error, ambient humidity, temperature and pressure.

According to the present invention, the above-mentioned reference profiles of the reflective membrane may be provided respectively in association with certain-order elements of Zernike polynomials.

Needless to say, it is possible to realize optical apparatus and ocular fundus observation apparatus which comprise any of these aberration correction apparatus.

According to another aspect of the present invention, there is provided a deforming method of a deformable mirror which comprises a plurality of electrodes and a reflective membrane faced to the electrodes and deformed to a certain profile by static voltages applied to the electrodes to correct the wavefront distortion of a light flux incident on the reflective membrane. This deforming method comprises the steps of: detecting light which originates from a subject and is reflected by the deformable mirror; as a database, preliminarily storing a certain number of voltage-by-electrode patterns each of which corresponds with a different reference profile of the reflective membrane and specifies each set of a voltage and an electrode to which the voltage is to be applied; and updating each data of the database as appropriate.

According to another aspect of the present invention, there is provided a deforming method of a deformable mirror which comprises a plurality of electrodes and a reflective membrane faced to the electrodes and deformed to a certain profile by static voltages applied to the electrodes to correct the wavefront distortion of a light flux incident on the reflective membrane. This deforming method comprises the steps of: detecting light which originates from a subject and is reflected by the deformable mirror; as a database, preliminarily storing a certain number of voltage-by-electrode patterns each of which corresponds with a different reference profile of the reflective membrane and specifies each set of a voltage and an electrode to which the voltage is to be applied; choosing a voltage-by-electrode pattern suited for a target detection signal to update the voltage-by-electrode pattern for the profile of the reflective membrane; and calibrating the deformation of the deformable mirror by updating each data of the database based on the updated pattern as appropriate.

According to another aspect of the present invention, there is provided an optical apparatus, comprising: a deformable mirror which comprises a plurality of electrodes and a reflective membrane faced to the electrodes and corrects the wavefront distortion of a reflected light flux incident on the reflective membrane which is deformed to a certain shape by applying static voltages to the electrodes; a storage section as a database to store a certain number of voltage-by-electrode patterns each of which corresponds with a different reference profile of the reflective membrane and specifies each set of a voltage and an electrode to which the voltage is to be applied; an aberration detecting section to detect the aberration of light which originates from a subject and is reflected from the deformable mirror; and an arithmetic and control section to calibrate the deformation of the deformable mirror by updating each data of the database as appropriate based on the detected aberration signal.

According to another aspect of the present invention, there is provided an optical apparatus, comprising: a deformable mirror which comprises a plurality of electrodes and a reflective membrane faced to the electrodes and corrects the wavefront distortion of a reflected light flux incident on the reflective membrane which is deformed to a certain shape by applying static voltages to the electrodes; a storage section as a database to store a certain number of voltage-by-electrode patterns each of which corresponds with a different reference profile of the reflective membrane and specifies each set of a voltage and an electrode to which the voltage is to be applied; an aberration detecting section to detect the aberration of light which originates from a subject and is reflected from the deformable mirror; and an arithmetic and control section which chooses a voltage-by-electrode pattern suited for a target detection signal stored in the storage section based on the aberration detection signal detected by the aberration detecting section and calibrates the deformation of the deformable mirror by updating the voltage-by-electrode pattern for the profile of the reflective membrane as appropriate based on the updated pattern.

The above-mentioned reference profiles of the reflective membrane may be provided respectively in the storage section in association with certain-order elements of Zernike polynomials.

It is possible to construct an ocular fundus observation apparatus which comprises any of these optical apparatus.

Thus, according to the present invention, although each deformable mirror has some specificity in terms of surface profile and deforming characteristics due to the manufacture/assembly and the stress in the mirror made of a SOI wafer and changes the surface profile and deforming characteristics as the operational environment changes in humidity, temperature and pressure, it is possible to eliminate the influence of the varying specificity of the individual deformable mirror. In addition, the deforming performance of the deformable mirror can be immune to the adjustment error which is inevitable when the total system is set up including the deformable mirror. Further, since the deformable mirror can be deformed as desired regardless of the environmental change in humidity, temperature and pressure, such measures as vacuum sealing of the deformable mirror are not necessary, resulting in lower manufacturing cost.

In addition, in an optical apparatus such as an ocular fundus observation apparatus, it is possible to use a Zernike voltage template adapted for aberration correction since each voltage template prepared for a Zernike order can be calibrated to the operational environment and specificity of the deformable mirror. Thus, accurate aberration correction can be attained.

Also in an aberration correction apparatus according to the present invention, although the formable mirror has some specificity in its surface profile and deforming characteristics due to the manufacture/assembly and the stress in the mirror made of a SOI wafer and changes the surface profile and deforming characteristics as the operational environment changes in humidity, temperature and pressure, it is possible to eliminate the influence of the varying specificity of the deformable mirror. In addition, the deforming performance of the deformable mirror can be immune to the adjustment error which is inevitable when the total system is set up including the deformable mirror. Further, since the deformable mirror can be deformed as desired regardless of the environmental change in humidity, temperature and pressure, such measures as vacuum sealing of the deformable mirror are not necessary, resulting in lower manufacturing cost.

Further, in an ocular fundus apparatus or the like according to the present invention, when the voltage template for the Zernike order of the aberration detection signal is calibrated to the specificity of the deformable mirror due to the manufacturing error, etc., the Zernike voltage template is approximated to the ideal one by iterating the process of updating the Zernike voltage template by using another Zernike voltage template chosen to get the Zernike voltage template closer to the ideal one. Each data of the Zernike voltage template database is updated as appropriate. Since the deformable mirror is deformed based on such a voltage template, correction values can be attained accurately and quickly.

### BRIEF DESCRIPTION OF THE DRAWINGS

FIG. 1 is a block diagram of an example of an ocular fundus observation apparatus to which a deforming method of a deformable mirror in accordance with the present invention is applied.

FIG. 2 is a block diagram of another example of an ocular fundus observation apparatus to which a deforming method of a deformable mirror in accordance with the present invention is applied.

FIG. 3 is a flowchart showing how a voltage template is calibrated.

FIG. 4 is a flowchart showing how a Zernike voltage template is calibrated in an ocular fundus observation apparatus in accordance with the present invention.

FIGS. 5(I), 5(II) and 5(III) show an example of creating voltage template Z(4, −4).

FIGS. 6(I), 6(II) and 6(III) show an example of creating voltage template Z(3, 1).

FIG. 7 shows initial voltage templates and the deformations of a deformable mirror caused by applying these templates respectively to the mirror.

FIGS. 8(I), 8(II) and 8(III) show that a surface profile is calculated by adding up Zernike coefficients and this calculation result agrees with the measurement result.

FIG. 9 is a cross-sectional view showing how a conventional deformable mirror is configured.

FIG. 10 is a cross-sectional view showing how the deformable mirror of FIG. 9 operates.

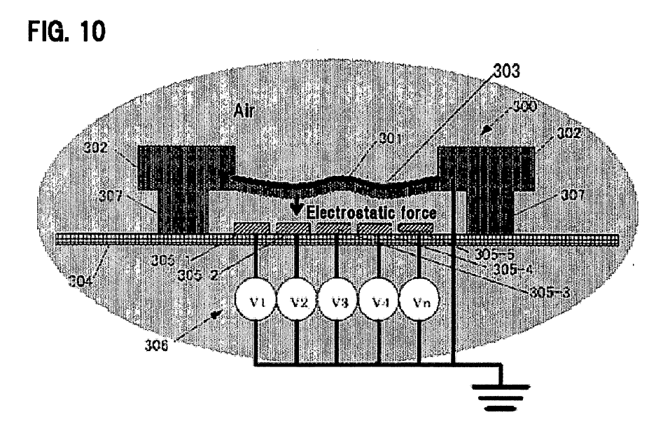

### DETAILED DESCRIPTION OF THE INVENTION

The following will describe the present invention with reference to the drawings. Note that identical parts in the drawings are referred to by a common reference number.

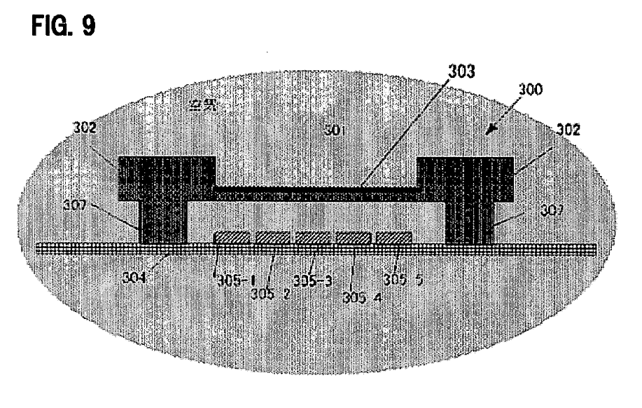

The present embodiment has a deformable mirror whose structure is similar to that of the mirror shown in FIG. 9 as a conventional example. That is, a deformable mirror 10 comprises: a membrane mirror fabricated by depositing an aluminum reflective film on a flexible Si membrane which is formed by etching a SOI wafer; a substrate kept a certain amount of distance apart from the membrane mirror by spacers disposed between the membrane mirror and the substrate; and electrodes disposed on the substrate. The present embodiment has an electrode array of 85 electrodes.

In addition, each electrode is connected to a power supply (not shown) similar to that of the conventional example. Certain voltages are respectively applied to the electrodes so that the part of the membrane mirror which faces each electrode is attracted by an electrostatic force which depends on the voltage V applied to the electrode and the distance between the membrane mirror and the electrode. Consequently, the membrane mirror is distorted or deformed as desired. Note that the present embodiment is structured so that an arbitrary voltage can be applied to each electrode.

Specifically, the deformable mirror may be fabricated as follows: The membrane mirror is formed by etching an SOI wafer of about 0.5 mm in thickness. Preferably, the membrane mirror has a thickness of several um. Its reflective portion is formed by vapor-depositing aluminum (Al), gold (Au) or the like on the top surface. In the present embodiment, the membrane mirror portion is electrically grounded. The substrate is made of glass, glass epoxy, Si or the like. The electrodes are formed by pasting thin metal sheets, such as Al ones. It is also possible to form the electrodes by vapor deposition of Au or the like. The spacers, which are, for example, high rigidity balls, are used to secure a certain gap between the membrane and the electrodes.

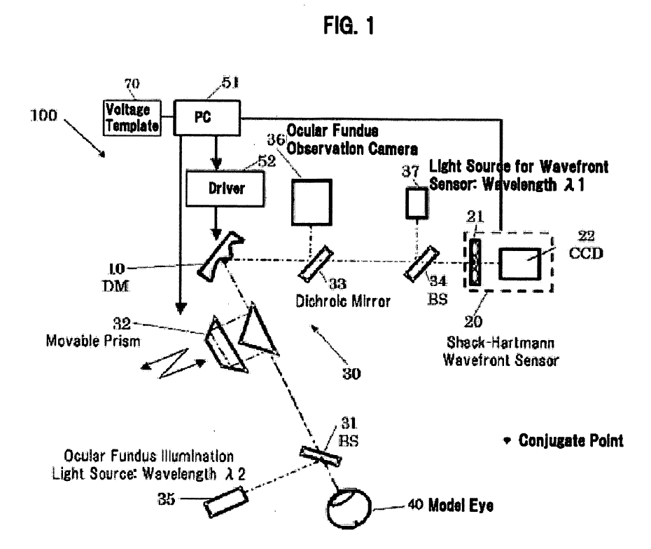

The following describes an ocular fundus observation apparatus using a deformable mirror in accordance with the present invention. FIG. 1 illustrates the ocular fundus observation apparatus of the present embodiment. This ocular fundus observation apparatus 100 is an adaptive optics system provided with a deformable mirror 10 and an aberration measuring device. The optical system of the ocular fundus observation apparatus in accordance with the present embodiment comprises the deformable mirror 10, a Shack-Hartmann wavefront sensor 20 to measure the eye's optical aberrations which occur in the crystal lens, retina, etc., and a beam transmission optical system 30.

When such an apparatus is used to observe the ocular fundus of a human eye, the fundus image may deteriorate due to the aberrations of the eye. The present embodiment is designed so that the deformable mirror 10 compensates for the aberrations to obtain a good fundus image. Also in the present embodiment, the voltage template for each Zernike order is calibrated according to the operational environment and the individual specificity of the deformable mirror by using an aberration-free model eye 40 at a position where a human eye is to be set during measurement of the human eye. That is, the aberration-free model eye is attached only when calibration is done. The beam transmission optical system 30 is provided with beam splitters 31 and 34, a movable prism 32, a dichroic mirror 33, an ocular fundus illumination laser diode 35 (wavelength λ2), a high sensitivity CCD 36 to pick up a fundus image, and a super luminescent diode (SLD) 37 (wavelength λ1) as a light source for the aberration measuring Shack-Hartmann wavefront sensor 40. By the dichroic mirror 33, the light (wavelength λ2) from the laser diode 35 is separated from the light (wavelength λ1) from the super luminescent diode (SLD) 37 which is the light source for the wavefront sensor.

The ocular fundus observation apparatus 100 of the present embodiment is also provided with a driver 52 to drive the deformable mirror 10. In addition, the present embodiment is provided with an arithmetic and control section (for example, a personal computer: PC) which receives a signal from the above-mentioned Shack-Hartmann wavefront sensor 20 and controls the driver 52 of the deformable mirror 10 and the movable prism 32 described later.

Further, a storage section to store calibration values necessary to calibrate a Zernike voltage template is provided in the PC 51 although not shown in the figure. In addition to this storage section, the PC 51 in the present embodiment is provided with a correction value calculating function. By this function, an ideal shape of the deformable mirror expected to be attained by applying voltages as specified in Zernike voltage template stored in the storage section is compared with a shape of the deformable mirror obtained by actually applying the voltages. From the differences, this function calculates correction values for voltages specified in the Zernike voltage template so that the deformable mirror can be deformed as desired.

In this ocular fundus observation apparatus 100, the length of the optical path between the SLD 37 and the wavefront sensor 20 can be adjusted by positioning of the movable prism 32 inserted in the optical path so that the light from the SLD 37 is converged on the ocular fundus 41. In this case, considering that when the light beam from the SLD 37 is reflected from the focal point on the ocular fundus 41, the signal peak of the reflected light received by the CCD should show its maximum, the prism is moved in a direction in which the signal peak increases until it reaches its maximum. In the present embodiment, the effective diameter of the deformable mirror 10 is 7.5 mm and the angle of incidence on the deformable mirror 10 is 15 degrees.

The SLD 37 and the wavefront measuring section, namely the wavefront sensor 20 of the widely known Shack-Hartmann type constitute a wavefront measuring system while the wavefront aberration measuring section and the computer 51 constitute a wavefront correction system. Comprising a Hartmann plate 21 (namely a microlens array) and a CCD 22, the wavefront aberration measuring section 20 receives the reflected light from the ocular fundus 41 and measures the wavefront aberration. The CCD 22 is placed at the focal point of the Hartmann plate. The reflection light from the ocular fundus 41 is converged on the CCD 22. The wavefront aberration appears as a displacement (Δx, Δy) of the point image on the CCD 22. The deformable mirror 10 and the Hartmann plate 21 are optically almost conjugate. Likewise, the SLD 37, the ocular fundus 41 and the CCD 22 are almost conjugate.

The light (wavelength λ1) from the SLD 37 illuminates the inside of the model eye 40 via the beam splitter 34, several lenses (not shown), the dichroic mirror 33, the deformable mirror 10 and the beam splitter 31.

It is desirable that the light from the wavefront sensor illuminating light source should have high spatial coherence and low temporal coherence. As an example, a super luminescence diode (SLD) is employed here which can serve as a high brightness point source. Note that the light source is not limited to a SLD. It is possible to use such a light source as a laser diode which provides high spatial coherence and high temporal coherence. In this case, its temporal coherence is appropriately reduced by inserting a rotary diffusing plate or the like. It is also possible to use a LED which provides low spatial coherence and low temporal coherence if it outputs a sufficient amount of light. For example, in this case, a pinhole or the like is inserted at the position of the light source along the optical path.

Following the optical path backward, the reflection light from the ocular fundus 41 of the model eye 40 illuminates the Shack-Hartmann wavefront sensor 20. The Shack-Hartmann wavefront sensor 20 outputs wavefront information to the arithmetic and control section 51. Based on this wavefront information, the arithmetic and control section 51 outputs a deformable mirror control signal to deform the deformable mirror 10.

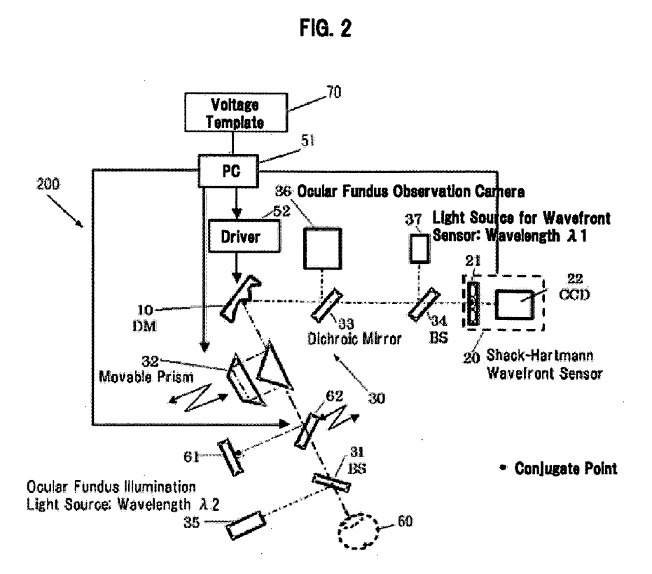

FIG. 2 shows another ocular fundus observation apparatus 200 which is functionally similar to the apparatus 100 of FIG. 1. Although the voltage template for each Zernike order is also calibrated according to the operational environment and the individual specificity of the deformable mirror, it is not necessary to attach or detach the aberration-free model eye 40 each time calibration is performed. This optical system is configured by adding a fixed mirror 61, a movable mirror 62 and a lens 63 to the optical system of the ocular fundus observation apparatus 100.

When an ocular fundus is observed, the movable mirror 62 is withdrawn from the optical path. When voltage templates are calibrated, the movable mirror 62 is inserted into the optical path. This positioning is controlled by the arithmetic and control section 51. The lens 63 is placed in the optical path toward the fixed mirror 61. This lens 63 is used to reverse the image which is sent to the Shack-Hartmann wavefront sensor 20 in the same manner as a fundus image when the ocular fundus is observed. This lens 63 and the fixed mirror 61, combined, are equivalent to the aberration-free model eye. The lens 63, the deformable mirror 10, the Hartmann plate 21 of the Shack-Hartmann wavefront sensor 20 are optically conjugate.

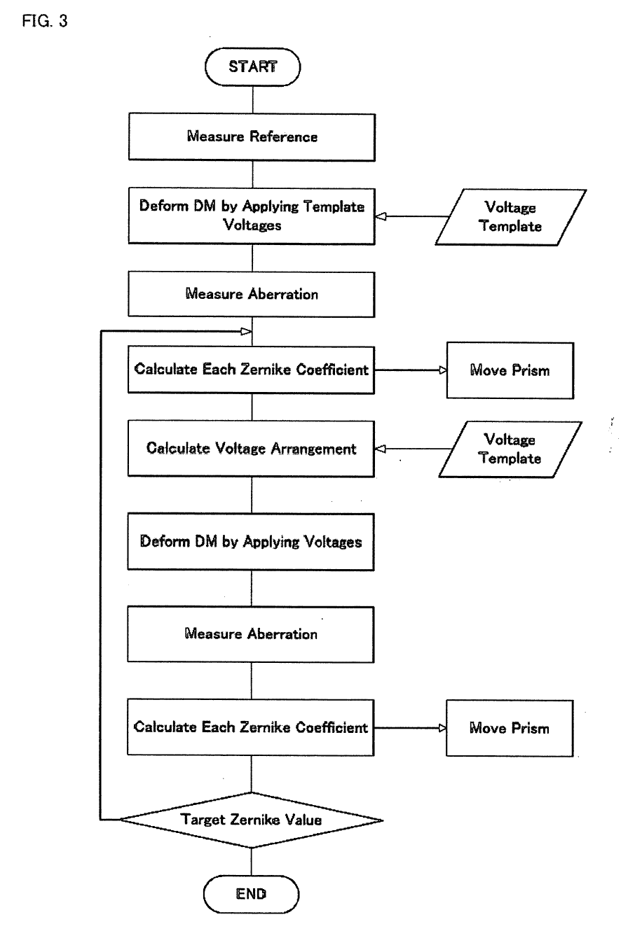

The following describes how a deformable mirror is deformed in an ocular fundus observation apparatus in accordance with the present embodiment. In the present embodiment, the voltage template for each Zernike order is calibrated according to the operational environment and the individual deformable mirror's specificity including the manufacturing error so that appropriate Zernike voltage templates can be used for aberration correction. FIG. 3 is a flowchart showing how voltage templates are calibrated in accordance with the present embodiment.

The aberration is expanded into Zernike polynomials as expressed below:
Total aberration=Σ zij×Z(i, j)
Where, Z(i, j) represents a given Zernike order and zi is the coefficient for it. An ideal voltage template for the order Z(i, j) is an arrangement of voltages which cause a non-zero coefficient value zi for the order Z(i, j) but no non-zero coefficient values for the other Zernike orders if applied to the deformable mirror.

The following describes a calibration procedure in accordance with the present embodiment. It is assumed that all Zernike order templates are already stored as initial values in a storage of the PC or the arithmetic and control section.

Firstly, in the ocular fundus observation apparatus 100 or 200 shown in FIG. 1 or 2, the reference aberration is measured (step S1 in FIG. 3: each step number hereinafter abbreviated as Sn (n=1, 2 . . . )). This measures the aberration which is caused by the non-deformed deformable mirror 10 and the beam transmission optical system. Then, a deforming voltage template (correction voltage table) for a given Zernike order is read out from the storage of the arithmetic and control PC where the voltage template is preliminarily stored. According to the voltage template, voltages are respectively applied to the electrodes 1 to 85 of the deformable mirror to deform the deformable mirror (step S2).

Then, after the aberration is measured (step S3), a coefficient is calculated for each Zernike order (step S4). Here, the initially measured reference aberration is subtracted from the newly measured aberration. Then, the movable prism is moved to correct the length of the optical path (step S5).

Then, from the voltage template (correction voltage table), a voltage arrangement for the electrodes 1 to 85 is calculated (Step S6). Then, the voltages are respectively applied to the electrodes 1 to 85 of the deformable mirror to deform the deformable mirror (step S7). The aberration is measured (Step S8) and each Zernike order coefficient is calculated (Step S9). Here again, the initially measured reference aberration is subtracted from the newly measured aberration. Then, the movable prism is moved to correct the length of the optical path (Step S10).

Then, comparison is made with the target Zernike coefficient value (S11). The procedure S6 through S10 is repeated until the Zernike coefficient becomes almost equal to the target value. This is performed for each Zernike order Z(i, j).

To calculate an arrangement of voltages to be applied to the respective electrodes of the deformable mirror, calculation is made according to the following equation:
Vn=sqrt((Σzij/zoij*Vij²)+Vn−1²)
where,

zij=zm−ztarget

Vn: n-times corrected applied voltage

zij: Desirable correction of Zernike coefficient value

zm: Measured Zernike coefficient value

ztarget: Target Zernike coefficient value

zoij: Coefficient value in Zernike voltage template

Vij: Template voltage

By performing this calculation for each of the electrodes 1 to 85, an arrangement of voltages to be applied to the respective electrodes is determined.

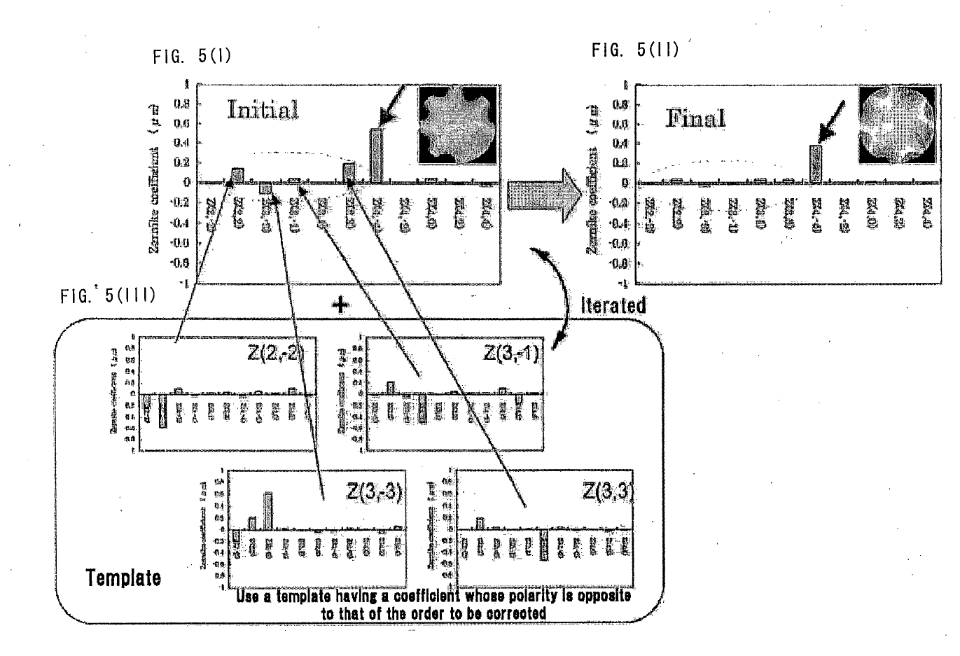

It is necessary to consider the polarities of coefficients for orders (unnecessary orders) other than the Zernike order of the template to be calibrated although this is omitted in the description of the above equation. For example, if the coefficient for an unnecessary Zernike order is a minus value, it is necessary to use a template which has a plus coefficient for the same order (Note that in FIG. 5, some templates have minus coefficients for a Zernike order while some have plus coefficients for the same Zernike order.)

As described above, the difference between measured Zernike coefficient value zm and target Zernike coefficient value ztarget is calculated to determine desirable correction zij of the Zernike coefficient value. Then, desirable correction zij is divided by coefficient value zoij in the Zernike voltage template and multiplied by the square of template voltage Vij. A new template voltage can be calculated from the summation of results obtained in this manner.

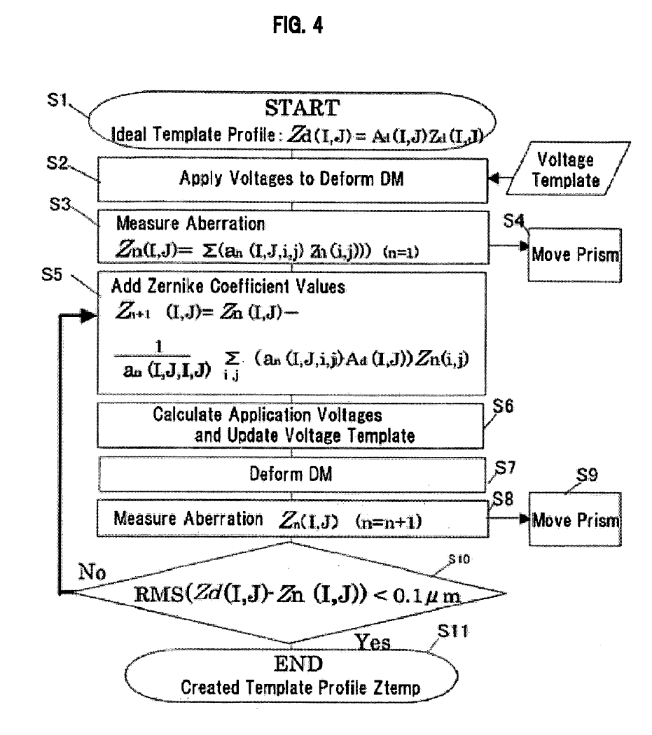

FIG. 4 is a more detailed flowchart showing how a Zernike voltage template is calibrated in the ocular fundus observation in accordance with the present invention.

In the present embodiment, the correction value calculation means performs calibration through the following procedure. It is assumed that all Zernike order templates are already stored as initial values in a storage of the arithmetic and control section 51 (for example a PC (Personal Computer)).

Firstly, the aberration-free model eye is set. The aberration is initially measured as the reference without applying voltages to the deformable mirror. This reference will be subtracted from each measured aberration. The ideal mirror profile to be attained by applying voltages of a Zernike voltage template to the deformable mirror is expressed as Zd(I, J)=Ad(I, J)Zd(I, J) wherein I and J represent Zernike order (±2, ±3, ±4, . . . ) and d means a design of an ideal voltage template.

Then, a Zernike voltage template 70 (correction voltage table) is read out from the storage of the arithmetic and control section 51 where the voltage template is preliminarily stored. According to the voltage template, voltages are respectively applied to the 85 electrodes Nos. 1 through 85) of the deformable mirror to deform the deformable mirror (S2).

The mirror surface profile is measured and calculated by using the wavefront sensor 20 and the computer 51 (S3). The measured surface profile, taken as the template profile, is given as below:
Zn(I, J)=Σ(an(I, J, i, j)zn(i, j))
n=1,
Where, an (I, J, i, j) is the coefficient for the zn(i, j) component. The ideal profile consists only of an(I, J, I, J)zn(I, J). The other components are noise components. The movable prism 32 is moved to compensate for the spherical component by correcting the length of the optical path (S4).

Noise components are removed from the measured surface profile Zn(I, J). Specifically, the difference between the ideal surface profile Zd(I, J) and the measured surface profile Zn(I, J) is obtained and the difference is subtracted from Zn(I, J) to obtain a deformation target profile Zn+1(I, J).

Zn(i, j) of the template profile Zn(I, J) is dimensionally different from Zd(i, j) of the ideal template profile Zd(I, J). Therefore, each voltage template to be superposed is normalized, namely, divided by coefficient an (I, J, I, J) (S5). Normalization (linearization) is done so as to eliminate undesirable Zernike orders from the voltage template. That is, the deformation target profile Zn+1(I, J), for which Zernike orders are to be added, is given by the equation below:
Zn+1(I, J)=Zn(I, J)−(1/an(I, J, I, J))Σ(an(I, J, i, j)−Ad(I, J))Zn(i, j))

Then, at (S5), a plurality of other voltage templates corresponding to Zernike orders necessary to attain voltages for the deformation target profile Zn+1(I, J) are selected and superposed after adjusted dimensionally. Voltages to be applied to the respective electrodes (Nos. 1 through 85) of the deformable mirrors are re-calculated accordingly to update the Zernike voltage template (S6).

Then, based on the updated voltage template, voltages are applied to the deformable mirror to deform it (S7) and measure the resulting surface profile Zn(I, J). At this time, increment (n=n+1) is done (S8). Then, the movable prism 32 is moved to correct the length of the optical path (S9).

Then, it is checked according to the following inequality whether the RMS difference between the measured surface profile Zn(I, J) and the ideal surface profile Zd(I, J) is smaller than a certain value, 0.1 μm in this case (S10):
RMS(Zd(I, J)−Zn(I, J))<0.1 μm
If the RMS value is larger than a certain value, the process is performed again from step S5. By repeating these processing steps (S5 through S10), a voltage template closer to the ideal voltage template is created.

By the above-mentioned iteration method, a Zernike voltage template adapted to the individual deformable mirror can be created from a preliminarily prepared voltage template. This makes it possible to improve the accuracy of deformable mirror-used aberration correction by eliminating the influence of the deformable mirror's specificity including manufacturing errors. In addition, since the voltage template is updated each time iteration is done, more accurate convergence is possible, resulting in a high accuracy Zernike voltage template.

With reference to FIGS. 5(I), 5(II), 5(III), 6(I), 6(II) and 6(III), the following exemplarily show how templates are prepared. FIGS. 5(I) to 5(III) show an example of preparing an ideal voltage template for the Zernike order Z(4, −4). Shown in FIG. 5(I) is a mirror profile Zn(4, −4) when voltages of an initial Z(4, −4) order Zernike voltage template are applied to the deformable mirror. It is assumed that the ideal mirror profile has 0.4 as the coefficient for the Z(4, −4) order. Therefore, Zd(4, −4)=−0.4*Zd(4, −4).

The actual Zn(4, −4) shows −0.01 μm for Z(2, −2), +0.15 μm for Z(2, 2), −0.1 μm for Z(3, −3), +0.02 μm for Z(3, −1), 0 μm for Z(3, 1), +0.18 μm for Z(3, 3), +0.55 μm for Z(4, 4), 0 μm for Z(4, −2), +0.02 μm for Z(4, 0), −0.01 μm for Z(4, 2) and −0.02 μm for Z(4, 4).

Other prepared Zernike voltage templates are selected for the initial voltage template as shown in FIGS. 5(I) to 5(III). Each selected Zernike voltage template has a Zernike coefficient whose polarity is opposite to the initial template's corresponding Zernike coefficient which is to be corrected. From the selected voltage templates, Zernike coefficients Z(2, −2), Z(3, −1), Z(3, −3) and Z(3, 3) are respectively picked out.

Since this process is to create an ideal Z(4, −4) template, the Zernike Z(2, −2) order voltage template to be added is divided by the aforementioned factor an(4, 4, 2, −2) so that it is matched dimensionally with the Z(2, −2) coefficient of the initial template.

Likewise, the Zernike Z(3, −1) order voltage template to be added is divided by the factor an(4, 4, 3, −1) so that it is matched dimensionally with the Z(3, −1) coefficient of the initial template. The Zernike Z(3, −3) order voltage template to be added is also divided by the factor an(4, 4, 3, −3) so that it is matched dimensionally with the Z(3, −3) coefficient of the initial template.

The Zernike Z(3, 3) order voltage template to be added is also divided by the factor an(4, 4, 3, 3) so that it is matched dimensionally with the Z(3, 3) coefficient of the initial template. After divided, these voltage templates are added to the initial voltage template to finally create a mirror profile Zn+1(I, J).

The Zernike template Zn(I, J) is replaced by the corrected mirror profile Zn+1(I, J). Then, the root mean square difference between the measured aberration sense signal's Zernike coefficients and the ideal Zernike coefficients is calculated. If the following inequality is not satisfied:
RMS(Zo(I, J)−Zn(I, J))<0.1 μm
the above mentioned calculation is iterated. If the RMS value is smaller than 0.1 μm, an ideal Z(4, 4) order Zernike voltage template is obtained.

Shown in FIG. 5(II) is a result of iterating the above-mentioned calculation. This voltage template has a coefficient value −0.005 μm for Z(2, −2), +0.02 μm for Z(2, 2), −0.01 μm for Z(3, −3), +0.01 μm for Z(3, −1), 0.05 μm for Z(3, 1), +0.05 μm for Z(3, 3), +0.4 μm for Z(4, 4), 0 μm for Z(4, −2), +0.01 μm for Z(4, 0), 0 μm for Z(4, 2) and −0.01 μm for Z(4, 4). That is, an ideal Z(4, −4) order voltage template is obtained.

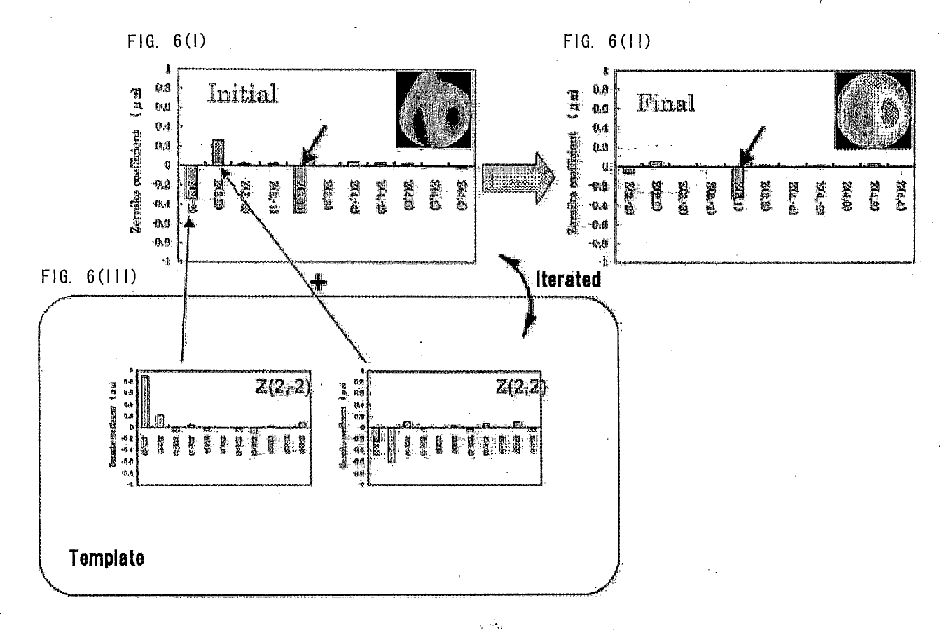

FIGS. 6(I), 6(II) and 6(III) show an example of preparing an ideal voltage template for the Zernike order Z(3, 1). Shown in FIG. 6(I) is a mirror profile Zn(3, 3) when voltages of an initial Z(3, 1) order Zernike voltage template are applied to the deformable mirror. It is assumed that the ideal mirror profile has 0.35 μm as the coefficient for the Z(3, 1) order. Therefore, Zd(3, 1)=−0.4*Zd(3, 1).

The actual Zn(4, −4) shows −0.35 μm for Z(2, −2), +0.25 μm for Z(2, 2), +0.01 μm for Z(3, −3), +0.01 μm for Z(3, −1), −0.5 μm for Z(3, 1), +0.01 μm for Z(3, 3), +0.03 μm for Z(4, 4), +0.02 μm for Z(4, −2), +0.02 μm for Z(4, 0), 0 μm for Z(4, 2) and −0.02 μm for Z(4, 4).

Other prepared Zernike voltage templates are selected for the initial voltage template as shown in FIG. 6(I). Each selected Zernike voltage template has a Zernike coefficient whose polarity is opposite to the initial template's corresponding Zernike coefficient which is to be corrected. From the selected voltage templates, Zernike coefficients Z(2, −2) and Z(2, 2) are respectively picked out. Since the current process is to create an ideal Z(3, 1) template, the Zernike Z(2, −2) order voltage template to be added is divided by the aforementioned factor an(3, 1, 2, −2) so that it is matched dimensionally with the Z(2, −2) coefficient of the initial template. Likewise, the Zernike Z(2, 2) order voltage template to be added is divided by the factor an(3, 1, 2, −2) so that it is matched dimensionally with the Z(2, 2) coefficient of the initial template. After divided, these voltage templates are added to the initial voltage template.

Shown in FIG. 6(II) is a result of iterating the above-mentioned calculation. This voltage template has a coefficient value −0.07 μm for Z(2, −2), +0.05 μm for Z(2, 2), +0.01 μm for Z(3, −3), −0.01 μm for Z(3, −1), −0.35 μm for Z(3, 1), +0.02 μm for Z(3, 3), −0.02 μm for Z(4, 4), +0.02 μm for Z(4, −2), +0.01 μm for Z(4, 0), +0.02 μm for Z(4, 2) and 0 μm for Z(4, 4). An ideal Z(3, 1) order voltage template is obtained.

The root mean square difference between the measured aberration sense signal's Zernike coefficients and the ideal Zernike coefficients was calculated and found to satisfy:
RMS(Zo(I, J)−Zn(I, J))<0.1 μm
That is, an ideal Z(4, 4) order Zernike voltage template is obtained.

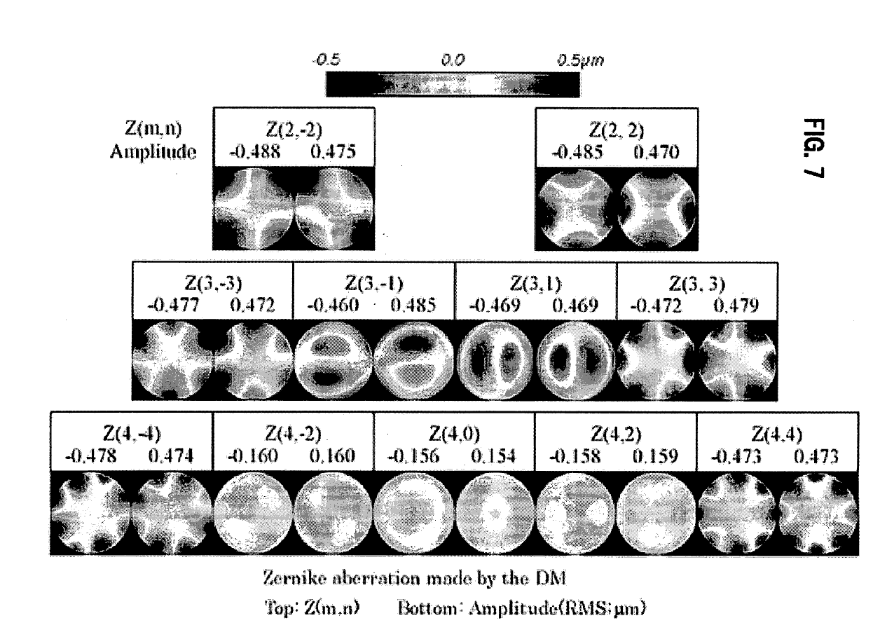

FIG. 7 shows each initial Zernike voltage template. The following describes a method employed to prepare these voltage templates. With certain voltages applied to the electrodes of the deformable mirror, the mirror profile is measured using a Fizeau interferometer. That is to say, the mirror's surface profile and each order Zernike coefficient are calculated from the interference fringes detected by the CCD of the Fizeau interferometer. In this example, Zernike aberrations made by the deformable mirror are limited to those of the fourth or lower orders. However, it is also possible to prepare Zernike voltage templates generating the fifth, sixth or still higher order Zernike aberrations. Since defocus can be corrected by the movable prism, the Z(2, 0) order template is not necessary. In addition, since the first order Zernike aberration corresponds to the tilt of the wavefront and therefore can be corrected by common optics not shown in FIGS. 1 and 2, the first order Zernike coefficients are not necessary.

Note that the computer program can be configured so that template calibration will automatically be started upon power on the ocular fundus observation apparatus. Although it is necessary to attach the aberration-free model eye 40 at first in the embodiment of FIG. 1, fully automatic template calibration is possible in the embodiment of FIG. 2 since the aberration-free model eye facility is incorporated in the apparatus. In addition, if sensors to detect the ambient humidity, temperature, pressure and so on are incorporated in the ocular fundus observation apparatus, in particular, near to the deformable mirror 10, it is possible to start template calibration when the environment remarkably changes. Since this makes the deformable mirror 10 not dependent on the environment, such a measure as vacuum sealing of the deformable mirror 10 must not be taken to eliminate its dependence on the environment. Therefore, the deformable mirror 10 can be made at lower cost since an expensive package or the like is not necessary.

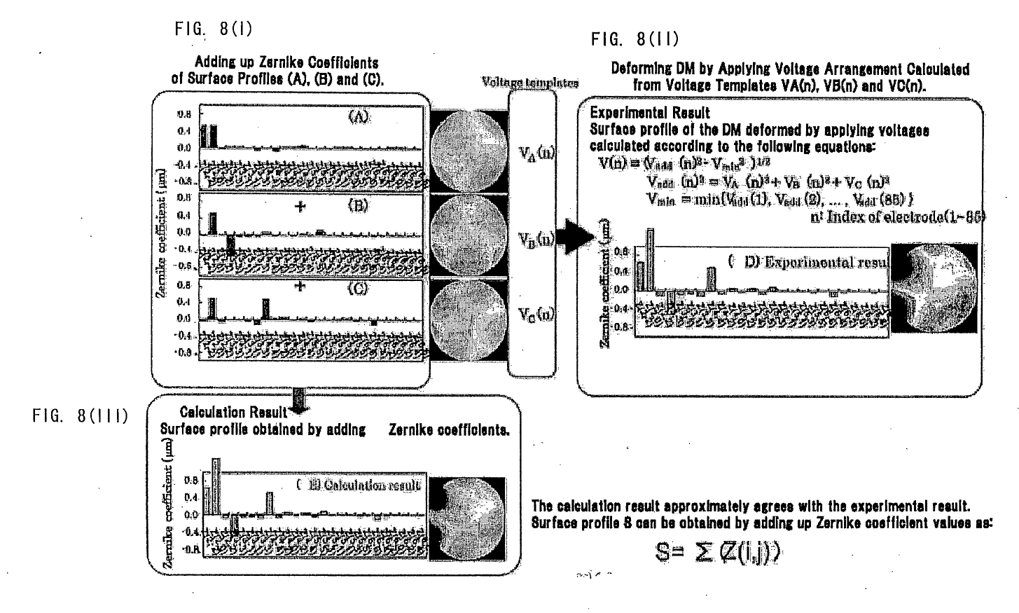

FIGS. 8(I), 8(II) and 8(III) show that a surface profile is actually calculated by adding up Zernike coefficients and this calculation result agrees with the measurement result.

In FIG. 8(I), surface profiles (A), (B) and (C) of the deformable mirror in terms of Zernike coefficients are shown with voltage templates VA(n), VB(n) and VC(n) which respectively correspond to the surface profiles. Shown in FIG. 8(II) is a surface profile (D) which was obtained by calculating an voltage arrangement from voltage templates VA(n), VB(n) and VC(n) shown in FIG. 8(I) and applying the voltage arrangement to the deformable mirror. Shown in FIG. 8(III) is a surface profile (E) calculated by adding up the Zernike coefficients.

In FIG. 8(II), voltages are determined according to the following equations:
V(n)=(Vadd(n)2−Vmin2)1/2
Vadd(n)2=VA(n)2+VB(n)2+VC(n)2
Vmin=min{Vadd(1), Vadd(2), . . . , Vadd(85)}

Symbol n represents an electrode number (1-85). In FIGS. 8(I) to 8(III), the surface profile (D) obtained by measurement agrees approximately with the surface profile (E) obtained by calculation. It is found that the surface profile S can be obtained by adding up Zernike coefficients as below:
S=Σ(Z(i, j))

In addition, the amplitude of a Zernike coefficient can be reduced by adding a Zernike coefficient value which is for the same order but opposite in polarity.

As described so far, the deformable mirror of the present invention can be deformed to an arbitrary surface profile by adding/subtracting ideal Zernike voltage templates. This can compensate for the aberration of a human eye.

To compensate for the aberration of a human eye, it is desirable to use an ideal Zernike voltage template which comprises only one Zernike order. If the mirror surface created according to a Zernike voltage template contains an undesired other Zernike order, its component serves as noise, making it impossible to accurately correct the aberration.

However, each deformable mirror has its own specificity in terms of shape and deforming characteristics due to such factors as manufacturing error. Therefore, using the same Zernike voltage template does not results in the same ideal surface profile defined only by one Zernike order but produces noise components. The present invention calibrates the Zernike voltage template so as to remove such noise components by adding/subtracting Zernike coefficients values as mentioned above. Thus, it is possible to implement an ideal deformation of the deformable mirror according to the calibrated voltage template.

The computer 51 in the present apparatus embodiment is provided with not only the aforementioned storage but also correction value calculation means by which the ideal surface profile of the deformable mirror to be attained by applying a Zernike voltage template stored in the storage is compared with the actual surface profile of the deformable mirror deformed by applying the Zernike voltage template and, from the difference, correction values for the Zernike voltage template are calculated so as to attain the desired surface profile.

In the above-mentioned method according to the present invention, any of the Zernike voltage templates prepared in the Zernike voltage template database may be modified iteratively in order to calibrate a Zernike voltage template. As compared with the method in which a prepared Zernike voltage template is modified according to the specificity including the operational condition, the present invention enables more accurate convergence, resulting in a high accuracy Zernike voltage template created.

In addition, it is possible to provide ocular fundus observation apparatus and other optical apparatus which employ the method of the present invention.

Note that although in the embodiment described so far, an ocular fundus observation apparatus is shown as an apparatus using a deformable mirror, the present invention can be applied to a variety of deformable mirror-used apparatus including head up displays, astronomical telescopes and laser illumination apparatus.

1. A deformable mirror deforming method in which wavefront distortion of a light flux reflected from a reflective membrane which is deformed to a certain shape is calibrated by applying static voltages to a plurality of electrodes disposed so as to face the reflective membrane, said deformable mirror deforming method comprising the steps of:
preliminarily storing a certain number of voltage-by-electrode patterns each of which is associated with a different reference profile of the reflective membrane and specifies each set of a voltage and an electrode to which the voltage is to be applied;
deforming the reflective membrane to a desired profile by mutually superposing said voltage-by-electrode patterns stored preliminarily; and
calibrating the deformation of the reflective membrane by correcting said certain number of voltage-by-electrode patterns.

2. A deformable mirror deforming method according to claim 1, wherein reference profiles of the reflective membrane are respectively provided in association with certain-order elements of Zernike polynomials.

3. A deformable mirror deforming method in which wavefront distortion of a light flux reflected from a reflective membrane which is deformed to a certain shape is calibrated by applying static voltages to a plurality of electrodes disposed so as to face the reflective membrane, said deformable mirror deforming method comprising the steps of:
preliminarily storing a certain number of voltage-by-electrode patterns each of which is associated with a different reference profile of the reflective membrane and specifies each set of a voltage and an electrode to which the voltage is to be applied;
deforming the reflective membrane to a desired profile by mutually superposing said voltage-by-electrode patterns stored preliminarily;
comparing a preliminarily stored detection signal with a detection signal detected actually later; and
calibrating the deformation of the reflective membrane by correcting the stored detection signal based on the actual operational condition of the deformable mirror so that a desired detection signal is obtained.

4. A deformable mirror deforming method according to claim 3 wherein said actual operational condition of the discrete deformable mirror includes at least one of the deformable mirror's manufacturing error, comprehensive setup error, ambient humidity, temperature and pressure.

5. A deformable mirror deforming method according to claim 1, wherein reference profiles of the reflective membrane are respectively provided in association with certain-order elements of Zernike polynomials.

6. An aberration compensation method for an optical apparatus, wherein:
the optical apparatus comprises a plurality of electrodes and a reflective membrane which is distorted by applying static voltages to the electrodes disposed so as to face the reflective membrane; and
a deformable mirror deforming method in accordance with claim 1 is performed as the aberration compensation method to correct the wavefront distortion of the reflected light flux from the reflective membrane of the optical apparatus.

7. An aberration compensation method for an ocular fundus observation apparatus, wherein:
the ocular fundus observation apparatus includes a plurality of electrodes and a reflective membrane which is distorted by applying static voltages to the electrodes disposed so as to face the reflective membrane; and
a deformable mirror deforming method in accordance with claim 1 is performed as the aberration compensation method to correct the wavefront distortion of a light flux reflected from the reflective membrane of the ocular fundus observation apparatus.

8. An aberration correction apparatus, comprising:
a deformable mirror which comprises a plurality of electrodes and a reflective membrane faced to the electrodes and calibrates wavefront distortion of a light flux reflected from the reflective membrane which is deformed to a certain shape by applying static voltages to the electrodes;
a storage section to store a certain number of voltage-by-electrode patterns each of which is associated with a different reference profile of the reflective membrane and specifies each set of a voltage and an electrode to which the voltage is to be applied;
an aberration detecting section to detect the aberration of light which originates from a subject and is reflected from the deformable mirror; and
calculation means for correcting the voltage-by-electrode patterns each of which is stored in the storage section, associated with a different reference profile of the reflective membrane and specifies each set of a voltage and an electrode to which the voltage is to be applied.

9. An aberration correction apparatus according to claim 8, wherein reference profiles of the reflective membrane are respectively provided in association with certain-order elements of Zernike polynomials.

10. An aberration correction apparatus, comprising:
a deformable mirror which comprises a plurality of electrodes and a reflective membrane faced to the electrodes and calibrates the wavefront distortion of a light flux reflected from the reflective membrane which is deformed to a certain shape by applying static voltages to the electrodes;
a storage section to store a certain number of voltage-by-electrode patterns each of which is associated with a different reference profile of the reflective membrane and specifies each set of a voltage and an electrode to which the voltage is to be applied;
an aberration detecting section to detect the aberration of light which originates from a subject and is reflected from the deformable mirror; and
correction value calculation means to compare a detection signal preliminarily stored in the storage section with an actual detection signal detected later to calculate correction values, wherein the correction values are used to correct the stored detection signal based on the actual operational condition of the deformable mirror so that a desired detection signal is obtained.

11. An aberration correction apparatus according to claim 10 wherein said actual operational condition of the discrete deformable mirror includes at least one of the deformable mirror's manufacturing error, comprehensive setup error, ambient humidity, temperature and pressure.

12. An aberration correction apparatus according to claim 11, wherein reference profiles of the reflective membrane are respectively provided in association with certain-order elements of Zernike polynomials.

13. An optical apparatus which is provided with an aberration correction apparatus according to claim 8.

14. An ocular fundus observation apparatus which is provided with an aberration correction apparatus according to claim 8.

15. A deformable mirror deforming method in which wavefront distortion of a light flux reflected from a reflective membrane which is deformed to a certain shape is calibrated by applying static voltages to a plurality of electrodes disposed so as to face the reflective membrane, said deformable mirror deforming method comprising the steps of:
detecting light which originates from a subject and is reflected by the deformable mirror;
as a database, preliminarily storing a certain number of voltage-by-electrode patterns each of which is associated with a different reference profile of the reflective membrane and specifies each set of a voltage and an electrode to which the voltage is to be applied; and
updating each data of the database as appropriate.

16. A deformable mirror deforming method in which wavefront distortion of a light flux reflected from a reflective membrane which is deformed to a certain shape is calibrated by applying static voltages to a plurality of electrodes disposed so as to face the reflective membrane, said deformable mirror deforming method comprising the steps of:
detecting light which originates from a subject and is reflected by the deformable mirror;
as a database, preliminarily storing a certain number of voltage-by-electrode patterns each of which is associated with a different reference profile of the reflective membrane and specifies each set of a voltage and an electrode to which the voltage is to be applied;
choosing a voltage-by-electrode pattern suited for a target detection signal to update the voltage-by-electrode pattern for the profile of the reflective membrane; and
deforming the deformable mirror by updating each data of the database based on the updated pattern as appropriate.

17. An optical apparatus, comprising:
a deformable mirror which comprises a plurality of electrodes and a reflective membrane faced to the electrodes and calibrates wavefront distortion of a light flux reflected from the reflective membrane which is deformed to a certain shape by applying static voltages to the plural electrodes;
a storage section as a database to store a certain number of voltage-by-electrode patterns each of which is associated with a different reference profile of the reflective membrane and specifies each set of a voltage and an electrode to which the voltage is to be applied;
an aberration detecting section to detect aberration of light which originates from a subject and is reflected from the deformable mirror; and
an arithmetic and control section to deform the deformable mirror by updating each data of the database based on the detected aberration signal as appropriate.

18. An optical apparatus according to claim 17, wherein reference profiles of the reflective membrane are respectively provided in the storage section in association with certain-order elements of Zernike polynomials.

19. An optical apparatus, comprising:
a deformable mirror which comprises a plurality of electrodes and a reflective membrane faced to the electrodes and calibrates wavefront distortion of a reflected light flux from the reflective membrane which is deformed to a certain shape by applying static voltages to the electrodes;
a storage section as a database to store a certain number of voltage-by-electrode patterns each of which is associated with a different reference profile of the reflective membrane and specifies each set of a voltage and an electrode to which the voltage is to be applied;
an aberration detecting section to detect aberration of light which originates from a subject and is reflected from the deformable mirror; and
an arithmetic and control section which chooses a voltage-by-electrode pattern suited for a target detection signal stored in the storage section based on aberration detection signal detected by the aberration detecting section and deforms the deformable mirror by updating the voltage-by-electrode pattern for the profile of the reflective membrane based on the updated pattern as appropriate.

20. An optical apparatus according to claim 19, wherein reference profiles of the reflective membrane are respectively provided in the storage section in association with certain-order elements of Zernike polynomials.

21. An ocular fundus observation apparatus provided with an optical apparatus according to claim 18.
